(19) United States
(12) Patent Application Publication
(10) Pub. No.: US 2025/0064454 A1
(43) Pub. Date: Feb. 27, 2025

(54) ANASTOMOSIS PROTECTION SYSTEM AND METHOD THEREOF

(71) Applicants: Jill Kelley, Tampa, FL (US); Scott T. Kelley, Tampa, FL (US)

(72) Inventors: Jill Kelley, Tampa, FL (US); Scott T. Kelley, Tampa, FL (US)

(21) Appl. No.: 18/237,890

(22) Filed: Aug. 24, 2023

(51) Int. Cl. A61B 17/11

(52) U.S. Cl. CPC A61B 17/1114; A61B 2017/00004; A61B 2017/1117

(57) ABSTRACT

The invention relates to a method, system, and device for protecting an anastomosis between at least two lumens of a body. The method includes identifying a requirement of creating the anastomosis between a first lumen and a second lumen of the at least two lumens of the body. The method further placing a tube made up of a biodegradable material, at a level of the anastomosis, inside the at least two lumens during the anastomosis or after the anastomosis, through a delivery system. It should be noted that the tube covers the anastomosis. The method further includes securing the tube at the level of the anastomosis to seal the tube against walls of the at least two lumens; and protecting the anastomosis by preventing a contact between the anastomosis and luminal contents associated with the at least two lumens of the body, through the tube.

### TECHNICAL FIELD

Generally, the invention relates to medical devices, and more particularly, to a method, system, and device for protecting an anastomosis between at least two lumens of a body through insertion of a tube.

### BACKGROUND

An anastomotic leak is a serious problem that may occur after surgery. The anastomotic leak may occur when a connection between two parts of a body (for example, two lumens) fails, and content inside the parts leak out through a joined area made by the surgery. This problem is one of the most serious complications of the surgery, arising difficulty in a recovery process. The seriousness of the anastomotic leak comes from the fact that interconnected pathways of the two parts of the body are naturally built to carry the contents in a specific and a separate way.

For example, an intricate ecosystem of a gastrointestinal tract, where the contents of digestion carry a medley of bacteria that are meant to reside solely within their designated confines. In such a case, in an event of an anastomotic leak along this convoluted pathway, these bacterial entities may seize opportunity to escape into areas where the bacterial entities are never supposed to be present. This unauthorized migration may lead to consequences, such as peritonitis or formation of abscesses, as the bacterial entities initiate infections upon encountering abdominal cavity.

To confront this critical issue, currently, medical professionals use a strategic defense mechanism, especially, in context of rectal anastomosis. The defense mechanism includes a temporary creation of an ostomy. The ostomy provides an alternative route for body waste to exit by skillfully rerouting a portion of the intestines (for example, by creating an opening). After some time, the ostomy may be reversed by closing the opening. Thus, the ostomy provides time to the joined area or anastomosis to heal properly. However, individuals who undergo an ostomy procedure may face various difficulties and challenges as they adapt to living with a stoma (i.e., the opening) and managing associated changes to their daily routines and lifestyles. For example, the difficulties may be associated with physical adjustments, stoma care, dietary changes, travel and activities, and the like.

The present invention is directed to overcome one or more limitations stated above or any other limitations associated with the known arts.

### SUMMARY OF INVENTION

In one embodiment, a method for protecting an anastomosis between at least two lumens of a body is disclosed. The method may include identifying a requirement of creating the anastomosis between a first lumen and a second lumen of the at least two lumens of the body. The method may further include placing a tube made up of a biodegradable material, at a level of the anastomosis, inside the at least two lumens during the anastomosis or after the anastomosis through a delivery system. It should be noted that the tube may cover the anastomosis. The method may further include securing the tube at the level of the anastomosis to seal the tube against walls of the at least two lumens. The method may further include protecting the anastomosis by preventing a contact between the anastomosis and luminal contents associated with the at least two lumens of the body, through the tube.

In another embodiment, an anastomosis protection system is disclosed. The anastomosis protection system may include an anastomosis protection device including a tube made up of a biodegradable material. The tube may be configured for protecting an anastomosis to be created between at least two lumens of a body by preventing a contact between the anastomosis and luminal contents associated with the at least two lumens. It should be noted that the tube may cover the anastomosis. The anastomosis protection system may further include a delivery system configured for placing the tube at a level of the anastomosis.

In yet another embodiment, an anastomosis protection device is disclosed. The anastomosis protection device may include a tube made up of a biodegradable material. The tube may be configured for protecting an anastomosis created between at least two lumens of a body by preventing a contact between the anastomosis and luminal contents associated with the at least two lumens. It should be noted that the tube may cover the anastomosis.

It is to be understood that both the foregoing general description and the following detailed description are exemplary and explanatory only and are not restrictive of the invention, as claimed.

### BRIEF DESCRIPTION OF THE DRAWINGS

The present application can be best understood by reference to the following description taken in conjunction with the accompanying drawing figures, in which like parts may be referred to by like numerals.

FIG. 1 illustrates a scenario of holding an anastomosis protection device configured for protecting an anastomosis, in accordance with an embodiment of the present disclosure.

FIG. 2 illustrates a scenario where an anastomosis protection device is placed between two lumens, in accordance with an embodiment of the present disclosure.

FIG. 3 illustrates an exemplary delivery system for placing an anastomosis protection device, in accordance with an embodiment of the present disclosure.

FIGS. 4A and 4B illustrate biodegradable stitches securing an anastomosis protection device, in accordance with an embodiment of the present disclosure.

FIG. 5 is a flowchart of a method of protecting an anastomosis between two lumens of a body, in accordance with an embodiment of the present disclosure.

FIGS. 6A-6C illustrate postoperative endoscopic views in different time periods, in accordance with an embodiment of the present disclosure, in accordance with an embodiment.

### DETAILED DESCRIPTION OF THE DRAWINGS

The following description is presented to enable a person of ordinary skill in the art to make and use the invention and is provided in the context of particular applications and their requirements. Various modifications to the embodiments will be readily apparent to those skilled in the art, and the generic principles defined herein may be applied to other embodiments and applications without departing from the spirit and scope of the invention.

Exemplary embodiments are described with reference to the accompanying drawings. Wherever convenient, the same reference numbers are used throughout the drawings to refer to the same or like parts. While examples and features of disclosed principles are described herein, modifications, adaptations, and other implementations are possible without departing from the spirit and scope of the disclosed embodiments. It is intended that the following detailed description be considered as exemplary only, with the true scope and spirit being indicated by the following claims. Additional illustrative embodiments are listed below.

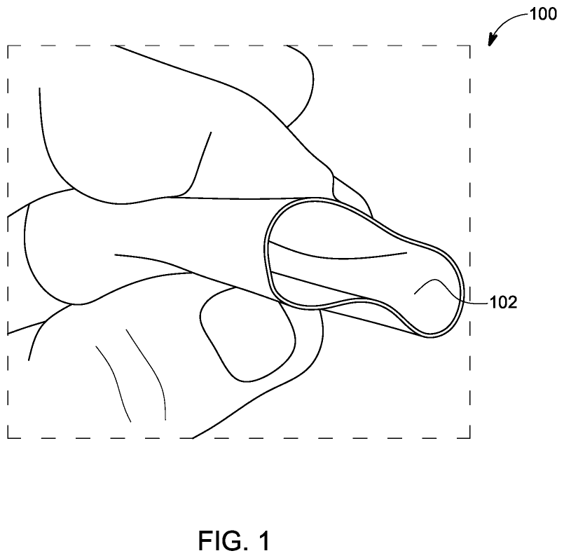

Referring now to FIG. 1, a scenario 100 of holding an anastomosis protection device 102 configured for protecting an anastomosis is illustrated, in accordance with an embodiment of the present disclosure. The anastomosis protection device 102 may corresponds to a tube (for example, a stent). Thus, the terms “anastomosis protection device”, “stent”, and “tube” are used interchangeably in the present disclosure.

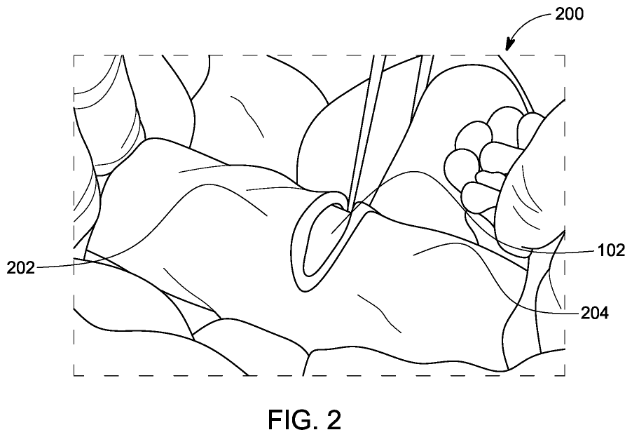

The anastomosis may be referred to as establishment of a connection, either through a surgery or naturally, between two tubular structures (such as lumens (as illustrated in FIG. 2)) within the body. The two tubular structures may include blood vessels, intestines, and other hollow organs. It may be apparent to a person skilled in the art that a primary objective of the anastomosis is to enable a seamless passage of fluids like blood or bodily waste between these the two interconnected tubular structures, ultimately leading to the restoration or enhancement of their operational capabilities.

The anastomosis protection device/stent/tube 102 may be made up of a biodegradable material. As the anastomosis protection device/stent/tube 102 is made up of biodegradable material, the anastomosis protection device/stent/tube 102 may further degrade within a predefined time-period of time (for example, 2 weeks, 3 weeks, 20 days, 12 days, and the like), depending on the biodegradable material used. In other words, the anastomosis protection device/stent/tube 102 may gradually break down and may be absorbed by the body, eliminating a need for its removal after its intended purpose is fulfilled.

Further, as illustrated in FIG. 1, the anastomosis protection device/stent/tube 102 may have a tube like structure with two open ends. Furthermore, the anastomosis protection device/stent/tube 102 may be an impermeable stent/tube which has an impermeable membrane. Therefore, the words stent/tube and impermeable stent/tube are used interchangeably in some embodiments of the present disclosure. The impermeable membrane of the stent/tube may not allow passage of fluids or contents associated with lumens to walls of the lumens. In other words, the stent/tube acts as a physical barrier against luminal contents.

Further, the anastomosis protection device/stent/tube 102 may protect and heal the anastomosis. To protect the anastomosis, the anastomosis protection device/stent/tube 102 may prevent a contact between the anastomosis and luminal contents associated with the lumens of the body. Thus, the anastomosis protection device/stent/tube 102 facilitates healing of the anastomosis by providing a physical barrier against the luminal contents.

The biodegradable material may be at least one of a polyaxial block copolymer, a Polylactic Acid (PLA), Polyglycolic Acid (PGA), a Poly Lactic-co-glycolic Acid (PLGA), a Polycaprolactone (PCL), or a Polyhydroxyalkanoates (PHA). For example, the anastomosis protection device/stent/tube 102 may be made up of Strateprene, which is a medical grade polyaxial block copolymer. The anastomosis protection device/stent/tube 102 may be available in different diameters (for example, 2 mm, 21 mm, 25 mm, 28 mm, 31 mm, 33 mm, and the like) and selected based on requirements and size and shape of the two tubular structures.

Strateprene has high elongation, high toughness, and lower modulus when compared with other polymers. Strateprene exhibits a coloration ranging from white to light tan granules. Further, inherent strength of the Strateprene is generally maintained for a period of 1 to 2 months, while its mass experiences a gradual reduction over the span of 6 to 9 months. The specific duration of strength retention and mass loss is contingent upon factors such as processing methods, physiological conditions, and the specific anatomical site of application. For example, Strataprene 5525 is composed of STRATAPRENE® 5525, material specifications, composition, item number (for example, 01404), appearance (for example, granules displaying a white to light tan coloration), granule size (for example, less than 4.0 mm), inherent viscosity (for example, a range from 1.1 to 1.8 dL/g), elemental composition (for example, 55% G, 25% TMC, 20% CAP), additional testing evaluated according to the standards of USP 232/233.

Strateprene may be packeged into 100 or 500 gm quantity. Standard packaging of the Strateprene includes an inner polythene bag, sealed inside a durable foil pouch to ensure protection from moisture and light. After opening the packaging, it may be recommended to protect the material from Ultraviolet (UV) light, in inert conditions, and under vacuum to maximize shelf life. When the material is stored in the original packaging at room temperature, the material may maintain its original properties for an extended period. The applications of the material may include Three Dimensional (3D) printing, injection molding, electrospinning, tubing, film extrusion, and the like.

In some embodiments the anastomosis protection device/stent/tube 102 may be made up of Strateprene and PGA. In such a case, the anastomosis protection device/stent/tube 102 not only contributes to protection of anastomosis but also to a healing process of the anastomosis. One of the remarkable and distinctive features of such biodegradable material is that the material includes a combination of Stratapene and/or PGA, lies in its exceptional capability to enhance the healing process in addition to providing reliable protection. This breakthrough is rooted in the inherent nature of these materials, which happen to be carbohydrates. This carbohydrate composition of the material brings about a host of advantages, particularly when adapted into a tube-like device for biomedical applications.

The utilization of biodegradable carbohydrates, as exhibited in the composition of Stratapene and PGA, presents an approach that addresses both the imperatives of protecting the anastomosis and fostering their healing. When implemented as a tube-like structure within the context of a medical device, this ingenious combination serves a dual purpose. The anastomosis protection device/stent/tube 102 may act as a safeguard against potential leaks, offering a robust physical barrier that prevents unwanted seepage. The biodegradable nature of the carbohydrates, namely Stratapene and PGA, further contributes to this protective function. Over time, the anastomosis protection device/stent/tube 102 begins to degrade gradually, allowing for a controlled breakdown that aligns with the body's natural healing processes. This measured degradation process ensures that the anastomosis protection device/stent/tube 102 retains its structural integrity for a defined period, effectively shielding the anastomosis from the risks of leakage throughout the crucial healing phase.

The carbohydrate composition, intricately designed to be biocompatible and biodegradable, interfaces seamlessly with the physiological environment. As the anastomosis protection device/stent/tube 102 begins to degrade, it releases bioactive compounds derived from the carbohydrate matrix. These compounds possess the inherent ability to stimulate cell proliferation and tissue regeneration in the vicinity of the anastomosis. Moreover, the carbohydrate-based composition encourages the recruitment of cells crucial to the healing process, such as fibroblasts and endothelial cells. These cells play pivotal roles in orchestrating the formation of new blood vessels, enhancing local perfusion, and ultimately expediting the healing process. The carbohydrates' carbohydrate-rich nature further complements the body's natural healing mechanisms, as carbohydrates are recognized to serve as essential energy sources for cellular activities and tissue repair.

In short, the anastomosis protection device/stent/tube 102 is not only used for protective qualities of the Stratapene and PGA-based material but also its strategic use as a conduit for healing. By integrating these materials into the anastomosis protection device/stent/tube 102, the benefits of carbohydrates may be used to create a biomedical solution that safeguards against leaks while actively promoting the intricate healing processes of anastomosis. This duality in function underscores the ingenuity of our proprietary material and its potential to significantly enhance patient outcomes in medical procedures involving anastomosis.

The anastomosis protection device/stent/tube 102 may be covered in a plastic envelope. Further, the plastic envelope may be enclosed with a foil pouch. The foil pouch serves as an additional layer of protection around the anastomosis protection device/stent/tube 102 and its plastic envelope. Prior to placing the anastomosis protection device/stent/tube 102 in a body, an entire package, i.e., the anastomosis protection device/stent/tube 102 within the plastic envelope enclosed in the foil pouch undergoes a sterilization. The sterilization is a crucial step to eliminate any potential microorganisms that may cause infections or complications when the anastomosis protection device/stent/tube 102 is implanted in the body. In the present disclosure, gamma radiation is used for the sterilization. The gamma radiation is a high-energy form of electromagnetic radiation. It effectively neutralizes bacteria, viruses, and other harmful microorganisms. By subjecting the entire package to the gamma radiation, any potential threats to patient safety are eliminated, ensuring that the anastomosis protection device/stent/tube 102 is free from contaminants.

Referring now to FIG. 2, a scenario 200 where the anastomosis protection device 102 is placed between two lumens (for example, a first lumen 202, and a second lumen 204) is illustrated, in accordance with an embodiment of the present disclosure. The first lumen 202 and the second lumen 204 are collectively referred to as lumens 202, 204. FIG. 2 is explained in conjunction with FIG. 1.

The lumens 202, 204 may include at least one of an organ or an anatomical structure. For example, the at least two lumens may include, but are not limited to, a rectum, a colon, a small intestine, an esophagus, an artery, a lung, a stomach, a ureter, a urethra, and blood vessels. The lumens 202, 204 may be, but is not to, in a gastrointestinal tract, a urinary tract, a cardiovascular system, a biliary tract, a genitourinary tract, and pancreatic duct. In FIG. 2, the lumens 202, 204 are two portions of intestine that may be joined after placing the anastomosis protection device/stent/tube 102 inside. However, other clinical applications of the anastomosis protection device/stent/tube 102 may also be contemplated.

Further, for example, the first lumen 202 corresponds to a first intestine portion and the second lumen 204 may correspond to a second intestine portion. With reference to the two open ends of the anastomosis protection device/stent/tube 102, one end of the two open ends may be inserted in an opening of the first lumen 202/first intestine portion, and another end of the two open ends may be inserted in an opening of the second lumen 204/second intestine portion. An inner diameter of the anastomosis protection device/stent/tube 102 may accommodate luminal content flow and unobstructed passage through the intestine.

In FIGS. 1-2, one shape, size and configurations of the anastomosis protection device/stent/tube 102 is illustrated for ease of explanation, however, other shapes of the anastomosis protection device/stent/tube 102 may also be considered based on requirements and lumen's shape, size and configurations. The shape may include, but is not limited to, a symmetrical shape, an asymmetrical shape, a cylindrical shape, a non-circular cross section, and the like.

It should be noted that the anastomosis protection device/stent/tube 102 may be placed using a delivery system. The anastomosis protection device/stent/tube 102 may be placed in such a way that the anastomosis protection device/stent/tube 102 may cover the anastomosis from inside when the anastomosis is created. For example, the anastomosis protection device/stent/tube 102 may be placed via at least one of a transanal placement and laparoscopic suturing, a surgical insertion placement, or a transabdominal placement.

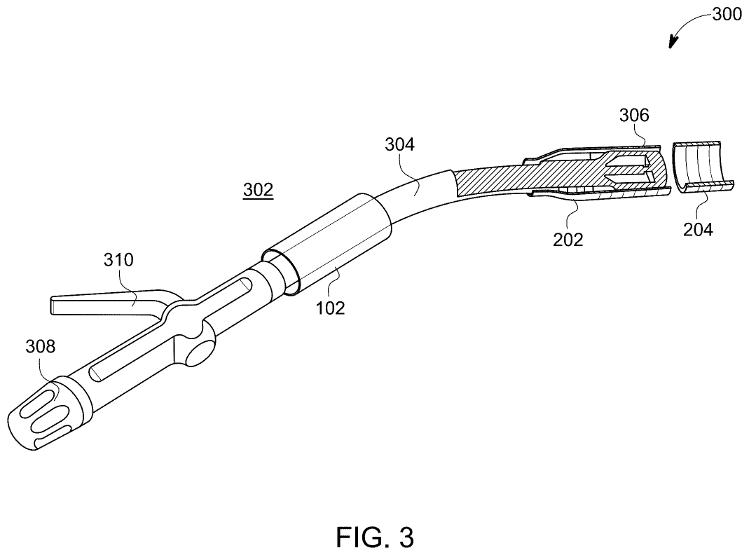

It may be apparent to a person skilled in the art that the delivery system includes a set of tools, instruments, and devices used to introduce and place a medical device (for example, the anastomosis protection device/stent/tube 102) inside the body. In particular, the delivery system may include at least one of an End-to-End Anastomosis (EEA) stapler, a rigid proctoscope, a sigmoidoscope, a colonoscope, or an endoscope detachably attached to the stent/tube through at least one of a detachable rigid tool or a detachable semirigid tool. The at least one of the detachable rigid tool or the detachable semirigid tool are adapted for advancing the stent/tube through the at least two lumens. The delivery system is further explained in detail in conjunction with FIG. 3.

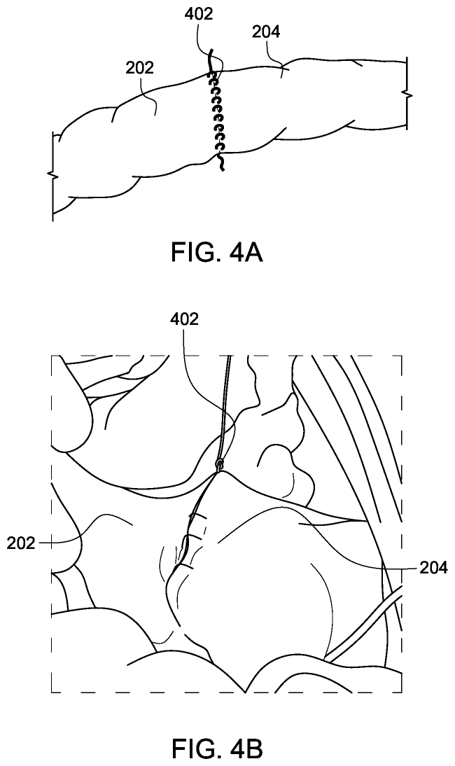

The anastomosis protection device/stent/tube 102 may be secured with the biodegradable fasteners or stitches, which is further explained and illustrated in detail in FIG. 4A and FIG. 4B. Once the anastomosis protection device/stent/tube 102 is secured, the anastomosis may be protected by preventing a contact between the anastomosis and luminal contents associated with the lumens 202, 204. Thus, anastomosis protection device/stent/tube 102 facilitates healing of the anastomosis by providing a physical barrier against the luminal contents. In some embodiments, a position of the anastomosis protection device/stent/tube 102 may be verified using an imaging technique. The imaging technique may be at least one of an endoscopy, a fluoroscopy, or an ultrasound.

Referring now to FIG. 3, the delivery system 300 is illustrated, in accordance with some embodiments of the present invention. FIG. 3 is explained in conjunction with FIGS. 1-2. The delivery system 300 may include at least one of an End-to-End Anastomosis (EEA) stapler, a rigid proctoscope, a sigmoidoscope, a colonoscope, or an endoscope detachably attached to the anastomosis protection device/stent/tube 102 through at least one of a detachable rigid tool or a detachable semirigid tool. The at least one of the detachable rigid tool or the detachable semirigid tool are adapted for advancing the anastomosis protection device/stent/tube 102 through the lumens 202, 204.

As illustrated in FIG. 3, the delivery system 300 includes an EEA stapler 302. The EEA stapler 302 may include a shaft 304, a head 306 present at an end of the shaft 304, a knob 308, and a lever 310. In some embodiments, the anastomosis protection device/stent/tube 102 may be carried on an outer surface of the shaft 304. The anastomosis protection device/stent/tube 102 may be advanced by an external pusher (not shown in FIG. 3), to an anastomosis level. In particular, the head 306 of the EEA stapler 302 may be inserted in the lumen 202 and advanced towards the lumen 204. Further, once the anastomosis level is reached, the EEA stapler 302 may be removed after leaving the anastomosis protection device/stent/tube 102 at the anastomosis level.

Referring now to FIG. 4A-4B, biodegradable stitches 402 securing the anastomosis protection device/stent/tube 102 are illustrated, in accordance with some embodiments of the present invention. FIGS. 4B-4B are explained in conjunction with FIGS. 1-3. The anastomosis protection device/stent/tube 102 may be secured at the level of the anastomosis. To secure the anastomosis protection device/stent/tube 102, at least one of biodegradable fasteners or biodegradable stitches (such as the biodegradable stitches 402) are used. As illustrated in FIGS. 4A and 4B, the anastomosis protection device/stent/tube 102 may be sealed with the biodegradable stitches 402 against walls of the lumens 202, 204.

The biodegradable stitches 402 are made up of at least one of a polyaxial block copolymer, a Polylactic Acid (PLA), Polyglycolic Acid (PGA), a Poly Lactic-co-glycolic Acid (PLGA), a Polycaprolactone (PCL), or a Polyhydroxyalkanoates (PHA). As the biodegradable stitches 402 are made up of biodegradable material, the biodegradable stitches 402 may further degrade within a predefined time-period of time (for example, 2 weeks, 3 weeks, 20 days, 12 days, and the like) depending on the biodegradable material used. In other words, the biodegradable stitches 402 may gradually break down and may be absorbed by the body, reducing the need for its removal after its intended purpose is fulfilled.

By way of an example, colorectal anastomosis for cancer may be performed using an EEA stapling device (such as the EEA stapler 302). The anastomosis protection device/stent/tube 102, may be designed to offer versatility in its size options, ensuring a secure fit within the lumen without exerting excessive tension on the surrounding wall. This approach aims to avoid any potential interference with the blood supply of the region while simultaneously ensuring a snug and accurate alignment with the colonic wall. The secure placement of the anastomosis protection device/stent/tube 102 is facilitated by employing biodegradable fasteners, as previously described.

To realize this objective effectively, the anastomosis protection device/stent/tube 102 may be constructed to closely align with the most frequently utilized diameters of the EEA Stapler, which is manufactured by companies. The ultimate vision for the anastomosis protection device/stent/tube 102 encompasses a comprehensive range of available sizes. This assortment is intended to cater to the diverse requirements of surgeons, taking into consideration the diameter of the colorectal anastomosis as well as the specific EEA stapler size employed for the anastomosis procedure. The approach mirrors a variety of choices currently offered for EEA staplers, which come in sizes such as 21 mm, 25 mm, 28 mm, 31 mm, and 33 mm.

In summary, the design philosophy behind the Leakguard device's sizing approach focuses on achieving optimal compatibility with the existing surgical tools and procedures. By emulating the available range of EEA stapler sizes, the Leakguard device aims to provide a comprehensive selection of sizes, catering to surgeons' preferences and specific clinical demands based on anastomosis diameters and stapler choices. This commitment to adaptability and precision underscores our dedication to enhancing surgical outcomes and patient care.

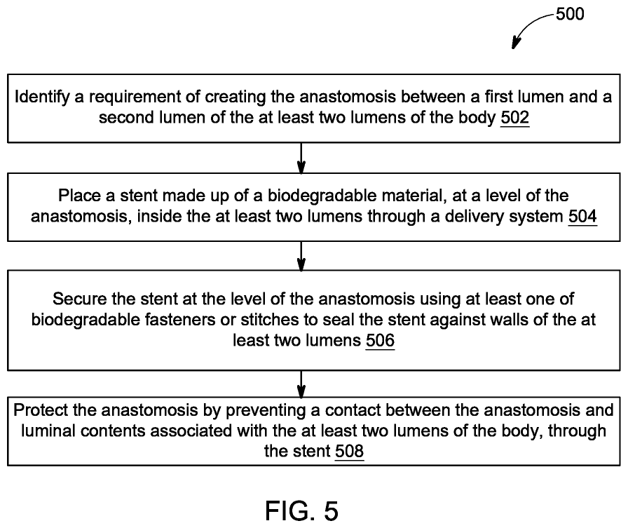

Referring now to FIG. 5, a method for protecting an anastomosis to be created between at least two lumens of a body is depicted via a flow chart 500, in accordance with an embodiment of the present disclosure. Each step of the flowchart 500 is performed using an anastomosis protection system. The anastomosis protection system may include an anastomosis protection device (for example the anastomosis protection device 100) and a delivery system (such as the delivery system 300).

At step 502, a requirement of creating the anastomosis between a first lumen and a second lumen of the at least two lumens (such as the lumens 202, 204) of the body may be identified. The at least two lumens may include at least one of an organ or an anatomical structure. For example, the at least two lumens may include, but are not limited to, a rectum, a colon, a small intestine, an esophagus, an artery, a lung, a stomach, a ureter, a urethra, and blood vessels.

The anastomosis may be referred to as establishment of a connection, either through a surgery or naturally, between two tubular structures (such as the at least two lumens) within the body. As already explained, the two structures may include blood vessels, intestines, and other hollow organs. It may be apparent to a person skilled in the art that a primary objective of the anastomosis is to enable a seamless passage of fluids like blood or bodily waste between these the two interconnected structures, ultimately leading to the restoration or enhancement of their operational capabilities.

With regards to requirement of the anastomosis, there may be many reasons that may include a surgical connection of the two tubular structures in the body. By way of an example, the anastomosis may be required for bypassing obstructions or blockages in blood vessels, intestines, or other hollow organs. In case of blocked coronary arteries, surgeons might perform coronary artery bypass grafting (CABG) to create anastomoses between a blocked artery and a healthy vessel, restoring blood flow to the heart muscle. By way of another example, the anastomosis may be required for organ transplantation. In organ transplantation, anastomosis is essential for connecting blood vessels and ducts of a transplanted organ to a recipient's body, ensuring proper blood flow, drainage, and function. For brevity, only two examples of requirement are explained, however there may be other examples where the anastomosis is required, for example, in treating injuries, managing diseases, addressing congenital abnormalities, creating access for dialysis, and the like.

Further, once the requirement is identified, at step 504, a stent/tube (i.e., the anastomosis protection device/stent/tube 102) made up of a biodegradable material may be placed or inserted, at a level of the anastomosis, inside the at least two lumens. The stent/tube may correspond to an impermeable stent/tube which has an impermeable membrane. Therefore, the words stent/tube and impermeable stent/tube are used interchangeably in some embodiments of the present disclosure. The impermeable membrane of the stent/tube may not allow passage of fluids or contents associated with the at least two lumens to walls of the at least two lumens. In other words, the stent/tube acts as a physical barrier against the luminal contents.

As the stent/tube is made up of biodegradable material, the stent/tube may further degrade within a predefined time-period of time (for example, 2 weeks, 3 weeks, 20 days, 12 days, and the like) depending on the biodegradable material used. In other words, the stent/tube may gradually break down and may be absorbed by the body, reducing the need for its removal after its intended purpose is fulfilled. The biodegradable material may be at least one of a polyaxial block copolymer, a Polylactic Acid (PLA), Polyglycolic Acid (PGA), a Poly Lactic-co-glycolic Acid (PLGA), a Polycaprolactone (PCL), or a Polyhydroxyalkanoates (PHA). For example, the stent/tube may be made up of Strateprene, which is a medical grade polyaxial block copolymer. The material Strateprene has high elongation, high toughness, and lower modulus when compared with other polymers.

For example, Strataprene 5525 is composed of STRATAPRENE® 5525, material specifications, composition, item number (for example, 01404), appearance (for example, granules displaying a white to light tan coloration), granule size (for example, less than 4.0 mm), inherent viscosity (for example, a range from 1.1 to 1.8 dL/g), elemental composition (for example, 55% G, 25% TMC, 20% CAP), additional testing evaluated according to the standards of USP 232/233.

In some embodiments, the stent/tube may be covered in a plastic envelope. Further, the plastic envelope may be enclosed with a foil pouch. The foil pouch serves as an additional layer of protection around the stent/tube and its plastic envelope. Prior to placing the stent/tube, the entire package—the stent/tube within the plastic envelope enclosed in the foil pouch undergoes a process called “sterilization”. The sterilization is a crucial step to eliminate any potential microorganisms that may cause infections or complications when the stent/tube is implanted in the body. In the present disclosure, gamma radiation is used for the sterilization. The gamma radiation is a high-energy form of electromagnetic radiation. It effectively neutralizes bacteria, viruses, and other harmful microorganisms. By subjecting the entire package to the gamma radiation, any potential threats to patient safety are eliminated, ensuring that the stent/tube is free from contaminants.

It should be noted that the stent/tube may be placed using a delivery system (such as the delivery system 300). The stent/tube may be placed in such a way that the stent/tube may cover the anastomosis from inside when the anastomosis is created. For example, the stent/tube may be placed via at least one of a transanal placement and laparoscopic suturing, a surgical insertion placement, or a transabdominal placement. It may be apparent to a person skilled in the art that the delivery system includes a set of tools, instruments, and devices used to introduce and place a medical device (for example, the stent/tube) inside the body. In particular, the delivery system may include at least one of an End-to-End Anastomosis (EEA) stapler, a rigid proctoscope, a sigmoidoscope, a colonoscope, or an endoscope detachably attached to the stent/tube through at least one of a detachable rigid tool or a detachable semirigid tool. The at least one of the detachable rigid tool or the detachable semirigid tool are adapted for advancing the stent/tube through the at least two lumens.

Thereafter, at step 506, the stent/tube may be secured at the level of the anastomosis. To secure the stent/tube, at least one of biodegradable fasteners or stitches (for example, the biodegradable stitches 402) are used. The stent/tube may be sealed with the at least one of biodegradable fasteners or stitches against walls of the at least two lumens. The at least at least one of biodegradable fasteners or stitches are made up of at least one of a polyaxial block copolymer, a Polylactic Acid (PLA), Polyglycolic Acid (PGA), a Poly Lactic-co-glycolic Acid (PLGA), a Polycaprolactone (PCL), or a Polyhydroxyalkanoates (PHA). As the at least at least one of biodegradable fasteners or stitches are made up of biodegradable material, the at least at least one of biodegradable fasteners or stitches may further degrade within a predefined time-period of time (for example, 2 weeks, 3 weeks, 20 days, 12 days, and the like) depending on the biodegradable material used. In other words, at least one of biodegradable fasteners or stitches may gradually break down and may be absorbed by the body, reducing the need for its removal after its intended purpose is fulfilled. Thus, the stent/tube facilitates healing of the anastomosis by providing a physical barrier against the luminal contents.

At step 508, the anastomosis may be protected by preventing a contact between the anastomosis and luminal contents associated with the at least two lumens of the body, through the stent/tube. Thus, the stent/tube facilitates healing of the anastomosis by providing a physical barrier against the luminal contents. In some embodiments, a position of the stent/tube may be verified using an imaging technique. The imaging technique may be at least one of an endoscopy, a fluoroscopy, or an ultrasound.

Strateprene has high elongation, high toughness, and lower modulus when compared with other polymers. Strateprene exhibits a coloration ranging from white to light tan granules. Further, inherent strength of the Strateprene is generally maintained for a period of 1 to 2 months, while its mass experiences a gradual reduction over the span of 6 to 9 months. The specific duration of strength retention and mass loss is contingent upon factors such as processing methods, physiological conditions, and the specific anatomical site of application. For example, Strataprene 5525 is composed of STRATAPRENE® 5525, material specifications, composition, item number (for example, 01404), appearance (for example, granules displaying a white to light tan coloration), granule size (for example, less than 4.0 mm), inherent viscosity (for example, a range from 1.1 to 1.8 dL/g), elemental composition (for example, 55% G, 25% TMC, 20% CAP), additional testing evaluated according to the standards of USP 232/233.

Strateprene may be packeged into 100 or 500 gm quantity. Standard packaging of the Strateprene includes an inner polythene bag, sealed inside a durable foil pouch to ensure protection from moisture and light. After opening the packaging, it may be recommended to protect the material from Ultraviolet (UV) light, in inert conditions, and under vacuum to maximize shelf life. When the material is stored in the original packaging at room temperature, the material may maintain its original properties for an extended period. The applications of the material may include Three Dimensional (3D) printing, injection molding, electrospinning, tubing, film extrusion, and the like.

In some embodiments the anastomosis protection device/stent/tube 102 may be made up of Strateprene and PGA. In such a case, the anastomosis protection device/stent/tube 102 not only contributes to protection of anastomosis but also to a healing process of the anastomosis. One of the remarkable and distinctive features of such biodegradable material is that the material includes a combination of Stratapene and/or PGA, lies in its exceptional capability to enhance the healing process in addition to providing reliable protection. This breakthrough is rooted in the inherent nature of these materials, which happen to be carbohydrates. This carbohydrate composition of the material brings about a host of advantages, particularly when adapted into a tube-like device for biomedical applications.

The utilization of biodegradable carbohydrates, as exhibited in the composition of Stratapene and PGA, presents an approach that addresses both the imperatives of protecting the anastomosis and fostering their healing. When implemented as a tube-like structure within the context of a medical device, this ingenious combination serves a dual purpose. The anastomosis protection device/stent/tube 102 may act as a safeguard against potential leaks, offering a robust physical barrier that prevents unwanted seepage. The biodegradable nature of the carbohydrates, namely Stratapene and PGA, further contributes to this protective function. Over time, the anastomosis protection device/stent/tube 102 begins to degrade gradually, allowing for a controlled breakdown that aligns with the body's natural healing processes. This measured degradation process ensures that the anastomosis protection device/stent/tube 102 retains its structural integrity for a defined period, effectively shielding the anastomosis from the risks of leakage throughout the crucial healing phase.

The carbohydrate composition, intricately designed to be biocompatible and biodegradable, interfaces seamlessly with the physiological environment. As the anastomosis protection device/stent/tube 102 begins to degrade, it releases bioactive compounds derived from the carbohydrate matrix. These compounds possess the inherent ability to stimulate cell proliferation and tissue regeneration in the vicinity of the anastomosis. Moreover, the carbohydrate-based composition encourages the recruitment of cells crucial to the healing process, such as fibroblasts and endothelial cells. These cells play pivotal roles in orchestrating the formation of new blood vessels, enhancing local perfusion, and ultimately expediting the healing process. The carbohydrates' carbohydrate-rich nature further complements the body's natural healing mechanisms, as carbohydrates are recognized to serve as essential energy sources for cellular activities and tissue repair.

In short, the anastomosis protection device/stent/tube 102 is not only used for protective qualities of the Stratapene and PGA-based material but also its strategic use as a conduit for healing. By integrating these materials into the anastomosis protection device/stent/tube 102, the benefits of carbohydrates may be used to create a biomedical solution that safeguards against leaks while actively promoting the intricate healing processes of anastomosis. This duality in function underscores the ingenuity of our proprietary material and its potential to significantly enhance patient outcomes in medical procedures involving anastomosis.

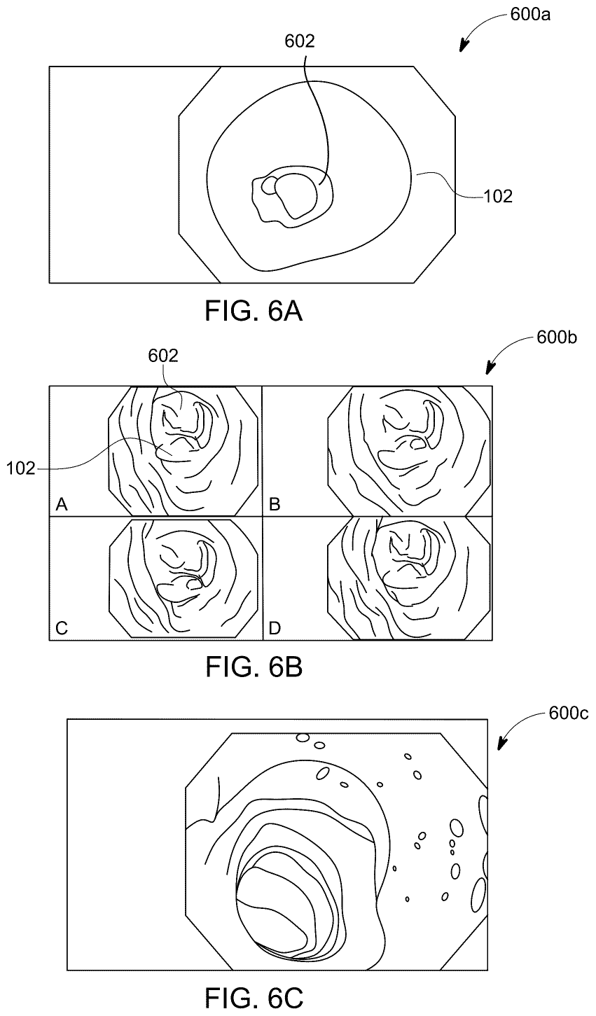

Referring now to FIGS. 6A-6C, postoperative endoscopic views 600A, 600B, 600C in different time periods are illustrated, in accordance with an embodiment of the present disclosure. In some embodiments, the anastomosis protection device/stent/tube 102 may be surgically placed inside rectum of pigs. Initially, a laparotomy may be performed, and the anastomosis protection device/stent/tube 102 may be placed directly (i.e., trans-abdominally) in the mid rectum of these animals after transecting the rectum. Further, the rectum may be closed with biodegradable sutures/stitches but left the anastomosis partially open to simulate an anastomotic leak. The anastomosis protection device/stent/tube 102 may be secured at place with the biodegradable sutures, at the proximal and distal ends of the device (placed from outside the colon), which anchored the anastomosis protection device/stent/tube 102 in place to both prevent migration and create a very effective seal to the colonic wall.

FIG. 6A illustrates an endoscopic view 600A of a freshly placed anastomosis protection device/stent/tube 102. The endoscopic view 600A shows a stool 602 travelling within the anastomosis protection device/stent/tube 102 with good colonic apposition. FIG. 6B illustrates an endoscopic view 600B after seven days of placing the anastomosis protection device/stent/tube 102. In the endoscopic view 600B, the stool 602 is within the anastomosis protection device/stent/tube 102, which means the anastomosis protection device/stent/tube 102 is still present and is not completely degraded (i.e., the stent/tube remained in a good position). Further, FIG. 6C illustrates an endoscopic view 600C after two weeks or fourteen days of placing the anastomosis protection device/stent/tube 102. The endoscopic view 600C represents degraded and disappeared the anastomosis protection device/stent/tube 102 with excellent healing of the anastomosis/site of potential leak. Postmortem analysis of these animals revealed no signs of inflammation, abscess, infection, or any other unexpected sign.

The results of this clearly shows success of the anastomosis protection device/stent/tube 102, and functioned as intended-without migration and effectively created a good seal against the colonic wall, and protected from anastomotic leak. In other words, the anastomosis protection device/stent/tube 102 effectively stayed in place, provided a successful bypass protecting the anastomotic area and continued to perform as intended. Additionally, the animals exhibited no sign of fever/tachycardia, and sepsis or evidence of peritonitis or abscess or inflammation at the anastomotic site or within the peritoneal cavity. The anastomosis protection device/stent/tube 102 stands to significantly decrease morbidity and mortality of anastomotic leaks after rectal resection.

As will be appreciated by those skilled in the art, the techniques described in the various embodiments discussed above are not routine, or conventional or well understood in the art. The techniques discussed above may provide several advantages over the prior art. The disclosure ultimately may help patients by eliminating need for an ileostomy, which is a current standard of care for anastomotic protection. The disclosure may change that standard of care and allow patients to resume a more natural postoperative lifestyle, more quickly with avoidance of the complications associated with ileostomies, which has complication rates and significant risks. The disclosure may also obviate the need for a second operation to reverse the temporary ileostomy, thus altering the risk of this terrible disease.

The anastomosis protection device 102 provides protection during the critical healing phase, then slowly dissolves and passes naturally over time. The anastomosis protection device 102 eliminates need for second surgery, preventing complications. The anastomosis protection device 102 obviates the need for a colostomy bag or any additional surgeries. Currently, the healthcare industry is staggering behind with environmental issues. The anastomosis protection device 102 uses environment-friendly medically approved biodegradable materials, the anastomosis protection device 102 is a surgical solution with zero carbon footprint and no ecological waste, using a clean technology with a sustainable supply chain that is scalable. The breakthrough medical technology represents an inflection point for both modern medicine and a way the healthcare industry advances climate change. The anastomosis protection device/stent/tube 102 is successfully designed, developed, and tested from the ground up with the planet in mind. The anastomosis protection device/stent/tube 102 may catalyze a green revolution in the healthcare industry. The anastomosis protection device/stent/tube 102 may help in avoiding risky repeat surgeries, lowering costs and saving lives.

In light of the above-mentioned advantages and the technical advancements provided by the disclosed method and system, the claimed steps as discussed above are not routine, conventional, or well understood in the art, as the claimed steps enable the following solutions to the existing problems in conventional technologies. Further, the claimed steps clearly bring an improvement in the functioning of the device itself as the claimed steps provide a technical solution to a technical problem.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide illustrative examples of the principles of this invention and its practical application to thereby enable one of ordinary skill in the art to utilize the invention in various embodiments and with various modifications as are suited to the particular use contemplated.

While various embodiments have been described above, it should be understood that they have been presented by way of example only, and not limitation. Thus, the breadth and scope of the present disclosure should not be limited by any of the above-described exemplary embodiments, but should be defined only in accordance with the following claims and their equivalents.

1. A method for protecting an anastomosis, by an anastomosis system, between at least two lumens of a body comprising:
identifying a requirement of creating the anastomosis between a first lumen and a second lumen of the at least two lumens of the body;
creating the anastomosis;
placing a monolithic impermeable tube composed of a biodegradable material, at a level of the anastomosis using a delivery system comprising one of a detachable rigid or semi-rigid tool as a guide to advance the monolithic impermeable tube within the at least two lumens after creating the anastomosis, the monolithic impermeable tube being pushed over an outermost surface of the delivery system and guided to cover the anastomosis;
securing, the monolithic impermeable tube in place covering the anastomosis using a plurality of biodegradable stitches at the proximal and distal ends of the monolithic impermeable tube, wherein each of the plurality of biodegradable stitches are placed trans-abdominally from outside the first lumen and the second lumen, each of the plurality of biodegradable stitches being passed directly through each of the first lumen and the second lumen to form a seal at the proximal and distal ends of the monolithic impermeable tube against each of the first lumen and the second lumen, and to secure the monolithic impermeable tube to the tissue of each of the first lumen and the second lumen, preventing an anastomotic leak and protecting the anastomosis by preventing luminal contents associated with the at least two lumens of the body from contacting the anastomosis and walls of the first lumen and the second lumen.

2. (canceled)

3. The method of claim 1, wherein the monolithic impermeable tube [,] and the at least one of biodegradable stitches degrade within a predefined time period, and wherein the monolithic impermeable tube comprises an impermeable membrane.

4. The method of claim 1, further comprising facilitating healing of the anastomosis by providing the monolithic impermeable tube as a physical barrier against the luminal contents.

5. The method of claim 1, wherein the delivery system comprises at least one of an End-to-End Anastomosis (EEA) stapler, a rigid proctoscope, a sigmoidoscope, a colonoscope, or an endoscope detachably attached to the monolithic impermeable tube through the at least one of a detachable rigid tool or a detachable semirigid tool, wherein the at least one of the detachable rigid tool or the detachable semirigid tool are adapted for advancing the monolithic impermeable tube through the at least two lumens.

6. The method of claim 1, wherein the biodegradable material comprises at least one of a polyaxial block copolymer, a Polylactic Acid (PLA), Polyglycolic Acid (PGA), a Poly Lactic-co-glycolic Acid (PLGA), a Polycaprolactone (PCL), a combination of the polyaxial block copolymer and the PGA, or Polyhydroxyalkanoates (PHA).

7. The method of claim 1, further comprising verifying a position of the monolithic impermeable tube using an imaging technique, wherein the imaging technique comprises an endoscopy, a fluoroscopy, or an ultrasound.

8. The method of claim 1, wherein the monolithic impermeable tube is placed via at least one of a transanal placement and laparoscopic suturing, a surgical insertion placement, or a transabdominal placement.

9.

10.

11.

12.

13.

14.

15.

16.

17.

18.

19.

20.
